(19) United States
(12) Patent Application Publication
(10) Pub. No.: US 2016/0198970 A1
(43) Pub. Date: Jul. 14, 2016

(54) METHOD FOR DETERMINING DIASTASIS TIMING USING AN MRI SEPTAL SCOUT

(71) Applicant: SUNNYBROOK RESEARCH INSTITUTE, Toronto (CA)

(72) Inventors: Garry Liu, Toronto (CA); Graham Wright, Toronto (CA)

(21) Appl. No.: 14/910,775

(22) PCT Filed: Jul. 31, 2014
(86) PCT No.: PCT/CA2014/050725

§ 371 Date: Feb. 8, 2016

Related U.S. Application Data
(60) Provisional application No. 61/867,513, filed on Aug. 19, 2013.

(51) Int. Cl. A61B 5/0456; G01R 33/567; A61B 5/055

(52) U.S. Cl. CPC A61B 5/0456; A61B 5/055; G01R 33/5673

(57) ABSTRACT

A new MRI imaging sequence, the Septal Scout, has been presented. This new technique can accurately determine the timing of diastasis windows for the purpose of cardiac gating in applications such as high-resolution coronary MRA. The Septal Scout acquires 1D MR images along the long-axis of the basal ventricular septum either through projection imaging or 2D excitations. Each acquisition produces a line of data along the ventricular septum. The acquisition is repeated over time to generate a time-map of Septal Scouts. The data from the Septal Scout time-map is processed to generate a velocity graph of an ROI near the basal septum. From this graph, the beginning and end of diastasis is determined. This timing information is available for use to facilitate cardiac gating in subsequent high-resolution MR angiography.

### FIELD

The present disclosure relates to the magnetic resonance imaging (MRI) of the heart. Specifically, the present disclosure relates to the determination of timing of cardiac-cycle phases to guide cardiac MRI.

### BACKGROUND

Currently, the slow data acquisition speed of cardiac magnetic resonance imaging (MRI) requires image acquisition to span multiple heartbeats in many applications involving the imaging of the heart. Under this circumstance, to prevent motion artifacts resulting from the heart beating during data acquisition, beat-to-beat data acquisitions need to be synchronized to the same stationary phase of the cardiac cycle. Typically, diastasis is the longest stationary period of the cardiac cycle; it occurs in between the periods of ventricular fast filling and atrial contraction during ventricular diastole (see FIG. 1). Because cardiac motion is periodic, image data acquired during diastasis over multiple heartbeats will appear to be acquired while the heart is still, provided that the relevant physiology of the imaging subject such as the heart rate remains the same during imaging. This is the principle behind prospective cardiac gating.

Typically, to perform prospective cardiac gating, the gating parameters need to be set prior to image acquisition. Ideal gating parameters, however, vary between subjects, and with heart rate. Therefore, calibration of gating parameters is desirable. For example, currently in MRI, a low spatial-resolution video of the 4-chamber view of the heart is acquired and used to determine the timing of the diastasis window, usually by a visual search for serial stationary frames. This approach, however, may produce gating errors on the order of tens of milliseconds due to limited temporal and/or spatial resolution of the calibration video. Since diastasis is preceded and succeeded by periods of significant ventricular motion, gating errors of tens of milliseconds may incur significant motion artifacts in high-resolution applications of cardiac imaging such as coronary angiography.

Liu et al. has demonstrated, using ultrasound and x-ray imaging, that long-axis motion and stasis of the basal ventricular septum accurately predict the motion and stasis of the coronary vasculature, respectively [1]. Septal motion-based cardiac gating is therefore more accurate than conventional ECG gating. It is desirable to have an MRI-based technique that measures septal motion to determine the cardiac gating parameters for cardiac MRI applications.

### SUMMARY

In embodiments disclosed herein, a method and system for determining the timing of diastasis using MRI cardiac imaging are disclosed. Tissue along the long-axis of a patient's ventricular septum is activated by the MRI and images are taken of a region of interest such that a time map of the MR images is produced. In a preferred embodiment, the region of interest is at the base of the septum and the images are generated by using a 1D steady-state free-precession pulse sequence or by 2D excitations. The images are then processed such that a velocity graph of points in the region of interest is generated over the course of at least a heartbeat.

The start and end times of the diastasis period is then determined. The start and end times are typically measured as a delay relative to the beginning of the heartbeat, typically chosen to be the onset of ventricular systole, which in turn is typically indicated by the R-peak of the ECG, a characteristic point determinable by someone skilled in the art of medical imaging. Therefore, in an embodiment, the ECG is used alongside the present disclosed method.

Upon locating the R-peak, the start and end times of the diastasis can typically be determined by finding, on the velocity graph, the period of low velocity in between the early and late ventricular filling peaks. Many methods are known to someone skilled in the art for determining this low velocity period. The method for selecting the diastasis period is non-specific to the present disclosure. In the preferred embodiment, the diastasis period is defined to be in between the first and last inflection points, respectively, enclosed by the early and late ventricular filling peaks of the velocity graph. The early and late ventricular filling peaks are determinable by someone skilled in the art. The inflection points are second derivative nulls representing the approach to and departure from the low velocity time period. In another embodiment, the diastasis period may be defined as the time period between the early and late ventricular filling peaks that fall below an arbitrary velocity threshold.

MR images may be generated using magnitude or phase data observed by the MRI detectors. In a preferred embodiment, diastasis is determined by intersecting findings over multiple heartbeats. In an embodiment, diastasis may be determined for a single heartbeat.

### BRIEF DESCRIPTION OF THE DRAWINGS

Embodiments will now be described, by way of example only, with reference to the drawings, in which:

FIG. 1 depicts a timing diagram of a typical cardiac cycle. The ECG (top) provides a time reference over the course of a single heartbeat (R-R interval) for different cardiac phases (bottom) and their associated left ventricular pressure and volume (middle). Adapted from [4].

FIG. 2 depicts an image in the 4-chamber long-axis plane. A Scout Plane (dashed white box) is prescribed along the septal wall; this plane is perpendicular to the 4-chamber long-axis plane. The Septal Scout is formed by the projection of the Scout Plane in the direction through the 4-chamber long-axis plane. The Septal Scout encodes long-axis displacements of the septum.

FIG. 3 shows a set of Septal Scouts over time. The vertical dashed line shows a Septal Scout at a point in time, which increases to the right. The dotted box shows a region-of-interest (ROI) spanning approximately 1 cm in depth near the basal ventricular septum. The data in the ROI is processed to produce displace and velocity of the basal septum.

FIG. 4 depicts an example displacement graph of the ROI from FIG. 3.

FIG. 5 depicts an example velocity graph of the ROI from FIG. 3. A typical diastasis period of near zero velocity is shown to occur in between ventricular filling phases (early filling by ventricular relaxation, and late filling by atrial contraction).

FIG. 6 describes a system algorithm for using the Septal Scout method to guide the acquisition of coronary MR angiography images over multiple heartbeats.

FIG. 7 depicts embodiments of the present disclosure for detecting ventricular systole instead of diastasis.

FIG. 8 shows sample images of a proximal right coronary artery stenosis obtained by x-ray angiography, MRI guided by the Septal Scout, and conventional MRI.

A further understanding of the functional and advantageous aspects of the disclosure can be realized by reference to the following detailed description and drawings.

### DETAILED DESCRIPTION

Various embodiments and aspects of the disclosure will be described with reference to details discussed below. The following description and drawings are illustrative of the disclosure and are not to be construed as limiting the disclosure. The drawings are not necessarily to scale. Numerous specific details are described to provide a thorough understanding of various embodiments of the present disclosure. However, in certain instances, well-known or conventional details are not described in order to provide a concise discussion of embodiments of the present disclosure.

As used herein, the terms, “comprises” and “comprising” are to be construed as being inclusive and open ended, and not exclusive. Specifically, when used in this specification including claims, the terms, “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the term “diastasis window” refers to the time period spanning ventricular diastasis; “imaging window” refers to the time period spanning image data acquisition; and, “cardiac gating” refers to the method of synchronizing the imaging window to the diastasis window on a heartbeat-to-heartbeat basis for the purpose of avoiding cardiac motion artifacts.

As used herein, the expression “electrocardiogram” (ECG) is the graphical output of an electrical measurement obtained over a period of time from a pair of non-overlapping electrodes placed on a person's body surface. The electrodes detect the electrical activity of the person's heart. Typically, for MRI, more than two electrodes are placed on the person's chest, providing multiple ECG signals, known as “ECG leads.”

As used herein, the expression “R-peak” refers to the signal deflection on the ECG that is (1) associated in time with the onset of ventricular systole; and (2) caused by a bioelectrical depolarization wave propagating through the ventricular myocardium as observed by the electrodes on the body surface. The R-peak is often used to mark the beginning of a heartbeat.

As used herein, the expression “steady-state free-precession (SSFP) pulse sequence” refers to an MRI pulse sequence where (1) the readout gradient comprises of a zeroth- and first-moment nulled waveform; and (2) the transverse magnetization reaches a non-zero steady state prior to the application of each excitation pulse.

As used herein, the expression “1 D SSFP pulse sequence” refers to an SSFP pulse sequence used in conjunction with a slice excitation, and no phase encode gradients. The resultant reconstructed MR image is a 1D line image corresponding to the in-plane projection of the excited slice.

As used herein, the expression “k-space” refers to the data acquisition space in the MR image acquisition process.

As used herein, the expression “reconstructed image” refers to the image formed by processing the k-space data. Typically, this image reconstruction process involves the Fourier transform. The reconstructed image is comprised of pixel values of the complex mathematical type:

I=A+Bi (Eq. 1)

where I is the image matrix of pixel values, and A and B are the real and imaginary components, respectively, of I.

As used herein, the expression “magnitude image” means an image composed of the magnitude of the reconstructed image:

I_{M}=|I|

I_{M}=\sqrt{A²+B²} (Eq. 2)

where I_{M} is the magnitude image.

As used herein, the expression “phase image” means an image composed of the phase of the reconstructed image:

$${I}_{ϕ} = arg ( I ) \\ {I}_{ϕ} = \{ \begin{matrix}{tan}^{- 1} ( \frac{B}{A} ) & if A > 0 \\ {tan}^{- 1} ( \frac{B}{A} ) + π & if A < 0 and B \geq 0 \\ {tan}^{- 1} ( \frac{B}{A} ) - π & if A < 0 and B < 0 \\ \frac{π}{2} & if A = 0 and B > 0 \\ - \frac{π}{2} & if A = 0 and B < 0 \\ in determinate & if A = 0 and B = 0\end{matrix} \qquad ( Eq . 3 )$$

where I_{φ} is the phase image.

As used herein, the expression “projection” as applied to an image means to reduce the typically two-dimensional image to a one-dimensional line image by summing the pixel intensities along one direction. For example, the magnitude projection of an image along the row-direction is the summation of all the magnitude pixel intensities by the columns of the image to form a single row of magnitude intensities.

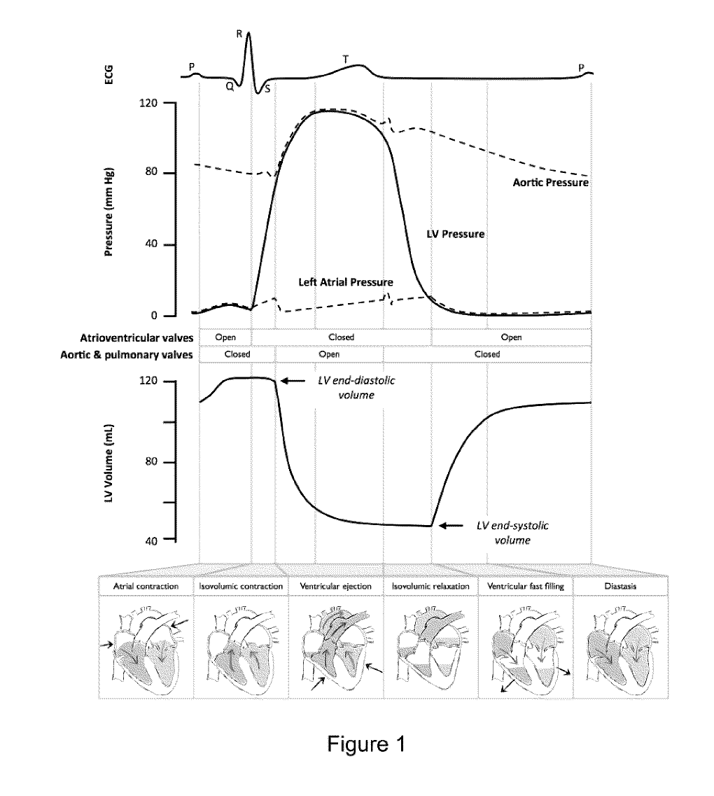

In medical imaging today, cardiac gating is commonly performed by referencing the electrocardiogram (ECG), a depiction of the electrical activity of the heart produced by measuring the voltage across pairs of electrodes placed on the chest. With reference to FIG. 1, the R-peak on the ECG is produced by the quickly propagating depolarization wave that triggers ventricular contraction, and the T-wave is produced by the subsequent slower repolarization process that accompanies ventricular relaxation. Isovolumic (ventricular) contraction, and ejection occur between the R-peak and the T-wave terminus; isovolumic relaxation, rapid filling, diastasis, and atrial contraction occur between the T-wave terminus and the R-peak. Since the R-peak is the most detectable feature of the ECG signal, it is used to mark the beginning of a cardiac cycle.

As used herein, the term “RR interval” refers to the time between two adjacent R-peaks on the ECG. It corresponds to the cardiac cycle duration, typically measured in milliseconds, and is inversely related to heart rate, typically measured in beats per minute.

As used herein, the term “trigger delay” refers to the time from the R-peak to the start of the imaging window.

As used herein, the term “gating parameters” refers to the trigger delay and imaging window duration.

As used herein, the term “gating error” refers to a misalignment between the imaging window and the diastasis window, causing a time difference between (a) the trigger delay and the beginning of the diastasis window, and/or (b) the imaging window duration and the diastasis window duration.

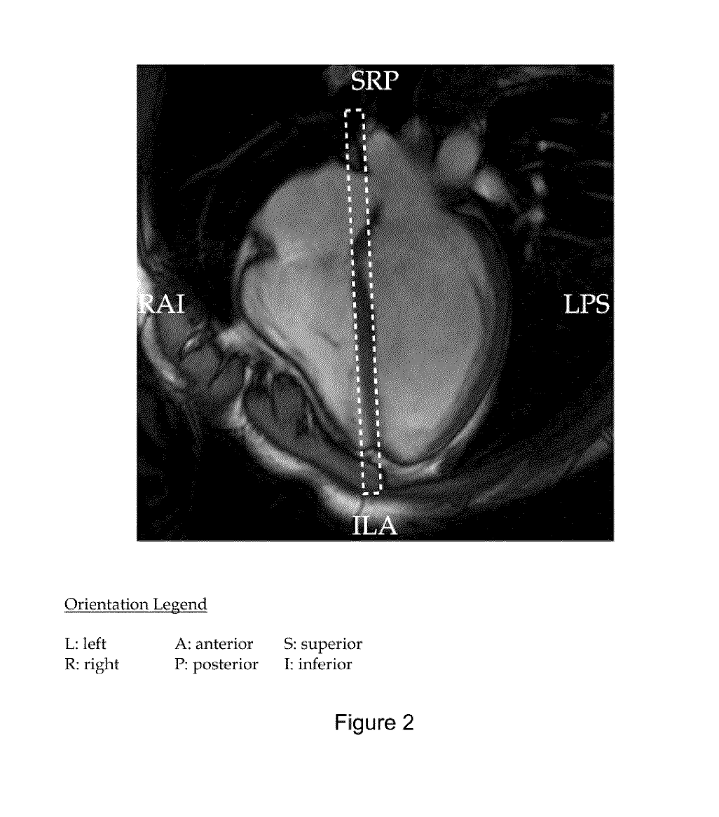

The present disclosure provides an MRI technique for determining the start and end of diastasis based on motion measurements of the ventricular septum. The technique provides line images, herein denoted “Septal Scouts,” that are magnitude projections of an image plane, herein denoted “Scout Plane,” which is oriented to be perpendicular to the 4-chamber long-axis plane and intersecting the approximate line formed by the septal wall, parallel to the long axis of the heart; the projection direction is through the 4-chamber long-axis plane (see FIG. 2). In the preferred embodiment, the Septal Scout image contrast is obtained by a 1D steady-state free-precession (SSFP) pulse sequence. In another embodiment, the Septal Scout image may be obtained by other 1D MR pulse sequences. It should be noted that the Septal Scout method is not limited to the SSFP family of pulse sequences, and that someone who is skilled in the art of MRI will be familiar with alternative sequences that although may provide different image contrast do not ultimately change the Septal Scout method itself.

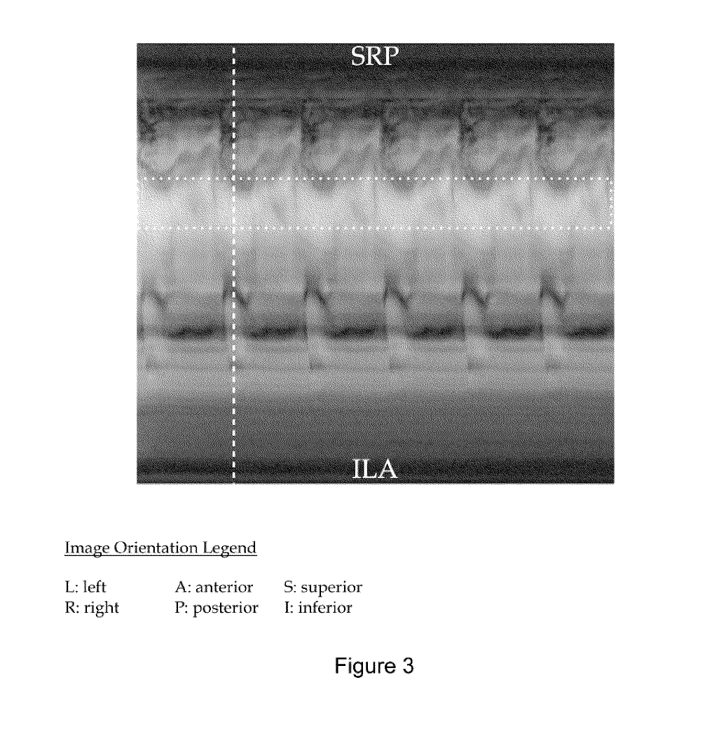

Referring now to FIG. 3, the Septal Scout line acquisitions (dotted white line) are repeated over time at a selectable temporal resolution on the order of milliseconds. In an embodiment of the present disclosure, the technique is performed during a breath hold, and respiratory motion is therefore negligible. Due to the fact that cardiac motion is the only dynamic component in the Scout Plane, the Septal Scouts encode long-axis displacements of the septum. The Septal displacement over time can be extracted from this Septal Scout time-map by analyzing the region-of-interest (ROI) spanning a small selectable depth range (approximately 1 cm) near the basal ventricular septum (dotted white box).

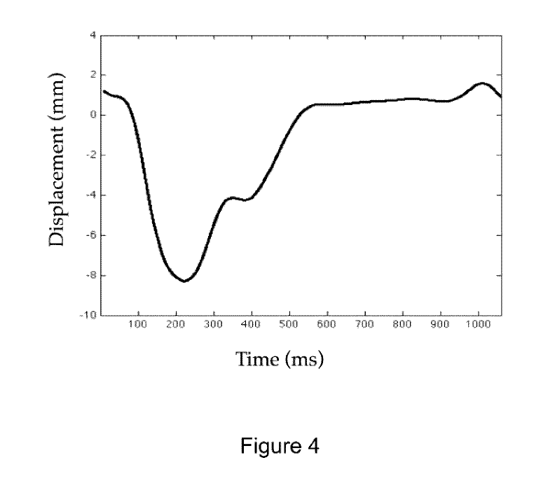

Referring now to FIG. 4, the set of Septal Scouts over time is processed to provide displacement measurements of an ROI spanning a small depth range (approximately 1 cm) near the basal ventricular septum. In one embodiment the displacement graph is obtained by first tracking an ROI on the first Septal Scout line to its displaced position on the second Septal Scout line. This is achieved by finding an ROI on the second Septal Scout line that provides the maximum correlation with the ROI on the first line, and recording the position of the tracked ROI on the second line. This process is repeated with successive pairs of Septal Scout lines to provide a step by step displacement graph of the basal ventricular septum. In an embodiment of the present disclosure, the displacement graph is obtained by averaging all the Septal Scout line intensities within a small depth range—about 1 cm—near the basal septum. The averaging operation suppresses image noise while it is assumed that the tissue within the small depth range moves approximately rigidly. Given the technique provides sufficient temporal resolution and that the Septal Scout line images have non-constant intensity patterns, this displacement graph can be differentiated in time to provide a velocity graph. This method operates on the principle that pixel intensity changes in the Septal Scout image is mainly caused by motion of the septum.

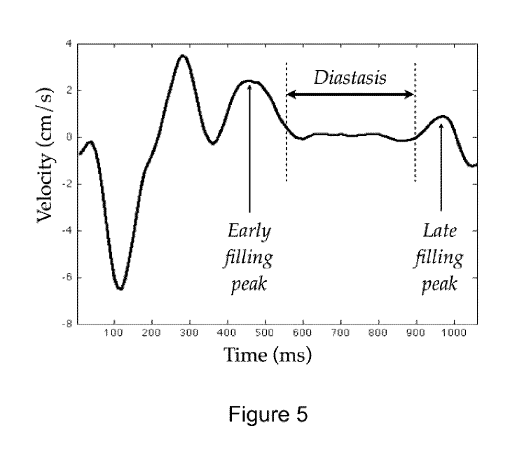

Referring now to FIG. 5, the displacement graph provided in FIG. 4 is processed to provide a corresponding velocity graph. In an embodiment, the displacement graph is differentiated in time to provide the velocity graph.

The velocity graph shows phases of ventricular dynamics and stases. It should be noted that the method for selecting the diastasis period is non-specific to the present disclosure. Many methods are known to someone skilled in the art. In the present embodiment, the start and end of diastasis is determined by the first and last inflection points (2nd derivative nulls), respectively, enclosed by the early and late ventricular filling peaks on the velocity graph. These characteristic time points represent the approach to and departure from the expected low velocity period enclosed in between early and late ventricular filling. In another embodiment, the start and end of diastasis may be determined by identifying a time period in between the early and late ventricular filling peaks during which the absolute value of the velocity function is below a selected threshold.

An embodiment of the present disclosure provides the use of two-dimensional (2D) excitation schemes. More specifically, the Septal Scout is no longer obtained by a one-dimensional projection of an excited Scout Plane. Rather, a 2D excitation pulse is used to excite a line or column of tissue at the intersection of the Scout Plane and the 4-chamber long-axis plane. The Septal Scout is then directly detected from the excited tissue. The cross-sectional shape of the column excitation is selectable, but is typically a circle. In addition, another 2D excitation scheme may be used. The Scout Plane and the 4-chamber long-axis plane may both be excited at half power, one immediately after the other; the two excited planes will produce a full power excitation at their intersection. The resultant Septal Scout will have a dominant signal source from the intersection of the two excited planes. The combination of excitation powers in this scheme is selectable.

An embodiment of the present disclosure provides the use of phase images in the Septal Scouts in addition to the conventional magnitude images. For example, the phase images of the Septal Scout are suitable for detecting accelerating blood or tissue where high intensities on the phase images represent high acceleration. This is described in more detail below in another embodiment of the present disclosure.

An embodiment of the present disclosure provides the determination of other cardiac phases, such as the end-systole period as an alternative cardiac gating window at high heart rates. End-systole is a low-cardiac-motion period that exists in between ventricular ejection and fast filling during the phase of isovolumic relaxation. It is typically shorter than diastasis, lasting less than 100 ms. In this embodiment, the Septal Scout velocity graph is used to identify a period of low velocity before the early ventricular filling peak. Specifically, the start and end of the end-systole period may be determined by identifying a time period before the early ventricular filling peak during which the absolute value of the velocity graph is below a selected threshold.

An embodiment of the present disclosure combines the Septal Scout technique with existing free-breathing MRI using respiratory navigators. To perform MRI during free-breathing, image data acquisitions are typically gated to the end-expiration phase of tidal breathing. Respiratory navigators are short MRI acquisitions that monitor the caudo-cranial position of the diaphragm, where end-expiration corresponds to the diaphragm being situated at the most caudal monitored position. In this embodiment, an MRA acquisition is performed during free-breathing. The Septal Scout is used to guide cardiac gating. At the same time, a respiratory navigator is used to identify the cardiac gating periods that occur during end-expiration. The data acquired during these coincident periods of cardiac and respiratory stasis are deemed free from motion artifacts and retained for reconstruction.

An embodiment of the present disclosure provides real-time acquisition of Septal Scouts such that the cardiac-gated imaging is triggered and terminated upon the real-time detection of the onset and end of diastasis, respectively. In this way, this implementation of the Septal Scout technique mimics a navigator approach. Furthermore, this embodiment precludes the use of the ECG for determining the imaging windows; rather, the R-peak of the ECG may be used to indicate the beginning of a pre-acquisition period during which contrast preparation such as fat-suppression may be performed.

An embodiment of the present disclosure provides an MRI-based cardiac gating system (MRI-CGS) based on the use of the Septal Scout. This system provides the benefit of not having to maintain an ECG signal to perform cardiac-gated MR imaging. Currently, the ECG signal may arbitrarily deteriorate due to loosened connections at the chest electrodes; also, R-peak detection may fail due to significant T-wave amplification. This system embodiment comprises of five functions:

Function 1: Calibration Scan
The system provides a gating window calibration scan. This scan performs Septal Scout acquisitions throughout a 20-second breath hold and determines, per heartbeat, diastasis start and end times relative to the corresponding systole onset. A multi-heartbeat imaging window that is intended to be compatible with the observed heart-rate variability (HRV) during the breath hold is then determined based on the intersection of the set of estimated diastasis windows. The approach here is to use smaller imaging windows to compensate for HRV in a multi-heartbeat acquisition. Alternatively, the system may aim to determine imaging windows in realtime during the same heartbeat as the image acquisition; this approach was not chosen to form the preliminary system design due to the associated practical limitations.

Function 2: Calibration Check
The system provides a calibration check at the beginning of each MRA acquisition. This scan applies the Septal Scout method during a heartbeat before the MRA scan. If the multi-heartbeat imaging window determined at calibration extends earlier and/or later beyond the diastasis window determined by the calibration check, the system indicates a need for recalibration of the gating parameters. This functionality attempts to detect when the MRI-CGS calibration has become obsolete. Typically, a change in the resting heart rate requires a recalibration.

Function 3: Ventricular Systole Detection
The system detects ventricular systole. Prospective gating requires a time reference at a consistent phase of the cardiac cycle for each heartbeat like the R-peak on the ECG, which marks the electrical onset of ventricular systole. Ventricular systole is a good candidate for the reference because (1) it typically occurs several hundreds of milliseconds before diastasis and therefore provides time for contrast preparation; and (2) it comprises of rapidly occurring events that are measurable such as ventricular depolarization, and ventricular ejection of blood into the aorta thereafter. The method of systole detection in the MRI-CGS may employ the ECG. In an embodiment detailed later, the Septal Scout is used to detect systole.

Function 4: Cardiac Gating
The system uses the gating window timing parameters determined during calibration, and limits imaging acquisition to that window every heartbeat. The system begins by performing the Septal Scout to identify systole onset in realtime, and then begins the count on the imaging trigger delay, which will synchronize acquisition to the beginning of diastasis. After acquiring data for the duration of the imaging window, the system will resume Septal Scout scans to look for the next occurrence of ventricular systole. The process repeats until the MRA acquisition is completed.

Function 5: Heart Rate Variability Tracking
The system monitors HRV. By tracking beat-to-beat ventricular systole, the system will monitor the variability of heartbeat durations (HBDs). For the nth heartbeat, failure to meet the condition, HBDₘᵢₙ<HBDₙ<HBDₘₐₓ will cause the system to indicate a need for recalibration; HBDₘᵢₙ and HBDₘₐₓ are the minimum and maximum HBDs detected, respectively, during the breath held calibration scan.
Along with tracking significant heart rate changes since the calibration scan, the system will monitor the HRV thresholds that have been shown by Leiner et al. to be effective buffers for maintaining coronary artery image quality against HRV [5]. For any n^{th} heartbeat during the MRA acquisition, the following condition is monitored: (HBDₘₑₐₙ−10%)<HBDn<(HBDₘₑₐₙ+50%), where HBDₘₑₐₙ is mean heart beat duration observed during calibration. Failure to meet this condition for all heartbeats will flag the scan for having high HRV. The user should consider this flag as a recommendation to reacquire the data.
This functionality is a check for significant changes in breath held heart rate patterns during the MRA acquisition beyond what was observed during calibration. It is also a check for a generally unstable heart rate that is a known cause for poor image quality.

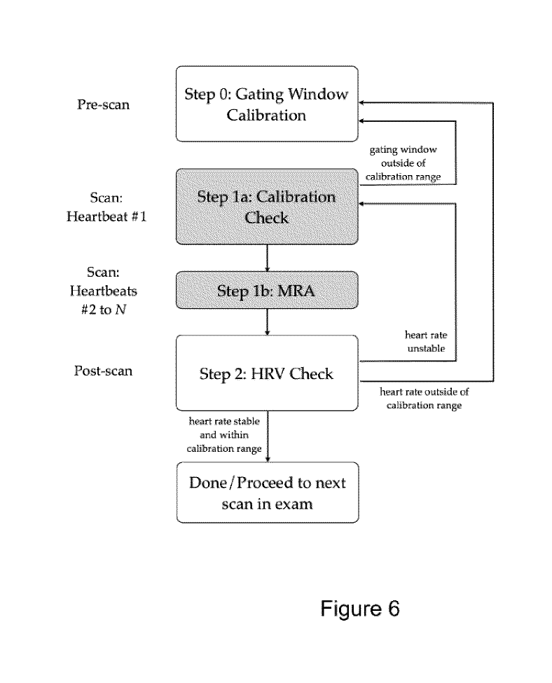

Referring now to FIG. 6, a flowchart is provided to illustrate the logical operations of the MRI-CGS system embodiment. Gating window calibration (Function 1) is performed at any point in the MRI study to calibrate the timing parameters of the estimated cardiac gating window to guide subsequent MRA acquisitions. Immediately before an MRA acquisition, a calibration check (Function 2) is performed to test whether a recalibration of the gating window is necessary. If not, the system proceeds to perform the MRA acquisition, which typically spans multiple heartbeats. The acquisition is therefore cardiac gated (Function 4). The HRV is tracked during the MRA acquisition, and an HRV check (Function 5) is performed to test whether the data needs to be reacquired. In an embodiment, the beginning of each heartbeat may be detected by the R-peak of the ECG (Function 3).

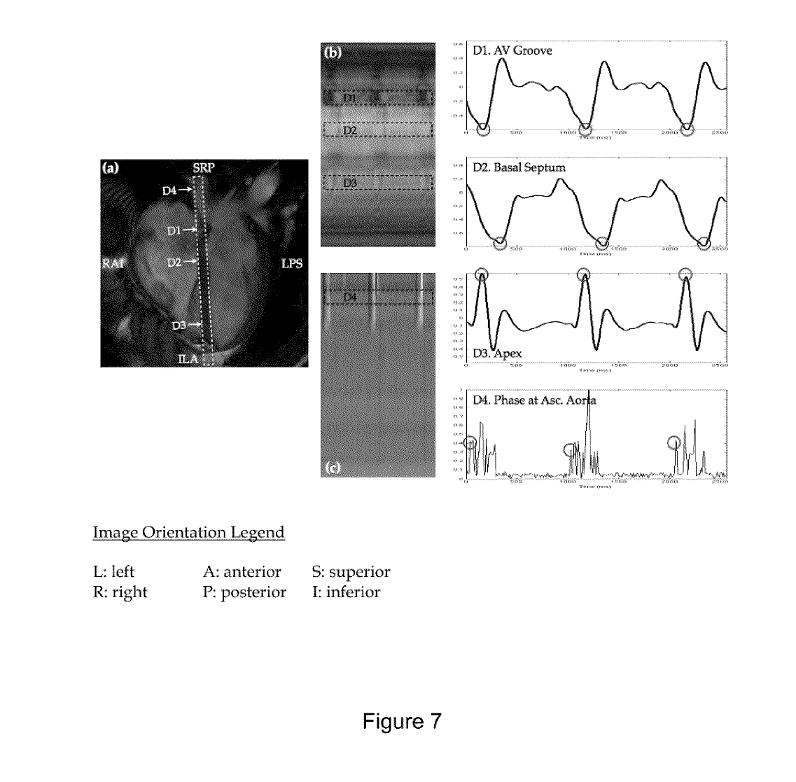

An embodiment of the present disclosure provides the detection of the onset of ventricular systole by monitoring the Septal Scout at depths that do not necessarily include the basal septum. Referring now to FIG. 7, a Septal Scout prescription similar to FIG. 2 is provided in (a) with the inclusion of the ascending aorta in the Scout Plane (dotted box). Four locations at different depths (D1, D2, D3, and D4) are shown. The Septal Scout time-map is shown in (b) spanning just over two heartbeats. Three displacement graphs are provided at D1, D2, and D3 for comparison from this map (dotted boxes). The phase-signal-version of the Septal Scout time-map is provided in (c). An absolute phase-intensity graph is provided at D4 near the ascending aorta by averaging the absolute phase intensities within D4. The corresponding displacement graphs for D1, D2, and D3, and the phase graph for D4 are shown in the right column. Note that an R-peak of the ECG corresponds to time zero, and at time 1023 ms. Red circles on each graph mark the supposed triggers that would represent the ventricular systole onset of each heartbeat according to the graph. This figure shows that there are various delays in systole detection relative to the R-peak by the different graphs. In terms of systole detection, D4 provides the least delay relative to the R-peak of the ECG, followed by an effective tie between D1 and D3, and then lastly, D2. Therefore, the Septal Scout provides several means for detecting the onset of ventricular systole.

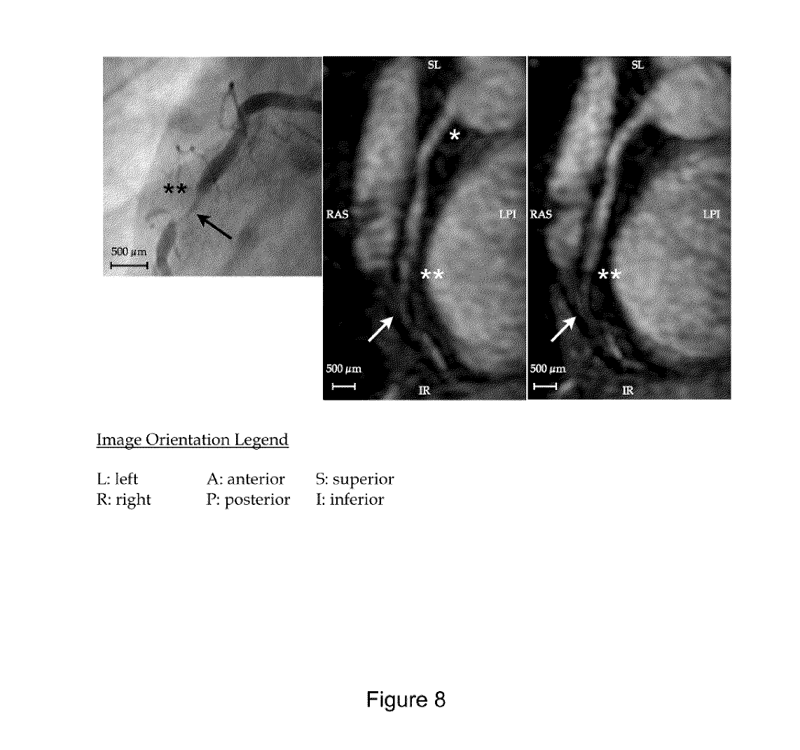

An embodiment of the present disclosure provides imaging of a coronary artery stenosis using the Septal Scout. Referring now to FIG. 8, a male patient with originally suspected, and later confirmed coronary artery disease was imaged using x-ray angiography, and MRA. A severe stenosis at the proximal right coronary artery is shown by an x-ray angiography image (left) and marked by a double asterisk. The corresponding MRA image that was acquired using a cardiac gating window identified by the Septal Scout method is shown (centre) with the stenosis marked by a double asterisk. The MRA image that was acquired using the conventional MRI technique where the cardiac gating window is identified by a cine-MRI sequence is shown (right) with the stenosis marked by a double asterisk. The Septal Scout-guided MRA image shows a more continuous tapering at the proximal entrance of the stenosis site compared with the cine-MRI guided image, and agrees better with the x-ray image.

The present Septal Scout technique can be clearly distinguished from MRI navigator techniques. In the past, MR projection imaging has been used to characterize one-dimensional motion of the diaphragm in respiratory navigator techniques [2], and lateral walls of the heart for cardiac navigator techniques [3]. The present disclosure can be distinguished from these previous navigator techniques by having a different target region of interest (ROI) for motion monitoring. The present disclosure focuses on the basal ventricular septum as a surrogate for motion of the coronary vasculature, as demonstrated by Liu et. al. [1]. The present disclosure is a novel use of MRI to track septal motion for the purpose of determining cardiac gating windows that is not obvious to one skilled in the art.

The foregoing description of the preferred embodiments of the invention has been presented to illustrate the principles of the invention and not to limit the invention to the particular embodiment illustrated. It is intended that the scope of the invention be defined by all of the embodiments encompassed within the following claims and their equivalents.

### REFERENCE

[1] G. liu, X.-L. Qi, N. Robert, A. J. Dick, and G. A. Wright, “Ultrasound-guided identification of cardiac imaging windows.,” Med. Phys. 39(6), 3009-3018 (2012) [doi:10.1118/1.4711757].
[2] M. A. Bernstein, K. F. King, and X. J. Zhou, “Handbook of MRI pulse sequences” (2004).
[3] P. Danias, K. Kissinger, and W. Manning, “Submillimeter Three-dimensional Coronary MR Angiography with Real-time Navigator Correction: Comparison of Navigator Locations1,” Radiology (1999).
[4] E. N. Marieb, M. D. P. D. Katja Hoehn, M. Hutchinson, J. Mallatt, and P. B. Wilhelm, Human Anatomy & Physiology, Benjamin Cummings (2012).
[5] T. Leiner, G. Katsimaglis, E. N. Yeh, K. V. Kissinger, G. Van Yperen, H. Eggers, W. J. Manning, and R. M. Botnar, “Correction for heart rate variability improves coronary magnetic resonance angiography.,” J. Magn. Reson. Imaging, vol. 22, no. 4, pp. 577-582, October 2005.

1. A method for determining the timing of diastasis using MRI cardiac imaging, comprising the steps of:
i) obtaining a magnetic resonance (MR) image along the long-axis of a ventricular septum;
ii) repeating the step (i) over time to generate a time-map of the MR images; and
iii) processing the data from the time-map to determine one or both of the beginning and an end of diastasis.

2. The method according to claim 1, wherein the step iii) comprises generating a position graph of a region of interest.

3. The method according to claim 2, wherein the step iii) comprises generating a velocity graph of the region of interest.

4. The method according to claim 3, wherein the velocity graph is generated by taking a first derivative with respect to time of the position graph of the region of interest.

5. The method according to claim 2, wherein the region of interest is near the base of the septum.

6. The method according to claim 2, wherein the region of interest spans a depth range of about 1 cm near the base of the septum.

7. The method according to claim 2, wherein the MR images are line images obtained by an MR pulse sequence.

8. The method according to claim 1, wherein the MR images are line images obtained by 2D excitations.

9. The method according to claim 1, wherein the step (i) is performed during a breath hold.

10. The method according to claim 1, wherein the beginning and the end of diastasis are determined relative to an R-peak.

11. The method according to claim 10, wherein the R-peak is determined by using an ECG.

12. The method according to claim 1, where the start and end times of diastasis are determined by finding time points that mark the approach to and departure from, respectively, a low velocity time period in between the velocity peaks associated with early and late ventricular filling.

13. The method according to claim 1, where the start and end of diastasis is determined by finding a time period in between the velocity peaks associated with early and late ventricular filling where the absolute velocity is below a selected threshold.

14. The method according to claim 1, wherein the magnetic resonance image is generated from phase data obtained from the MRI.

15. The method according to claim 1, wherein the magnetic resonance image is generated from magnitude data obtained from the MRI.

16. The method according to claim 1, wherein an overall diastasis window is determined by intersecting the diastasis windows from multiple heartbeats.

17. The method according to claim 1, wherein an overall diastasis window is determined to be the period in between the average start and end times of the diastasis windows from multiple heartbeats.

18. The method according to claim 1, wherein heart rate is observed such that an observation of a predetermined threshold of heart rate change indicates that a new timing of diastasis must be determined.

19. A system for determining the timing of diastasis using MRI cardiac imaging, comprising:
an MRI programmed for the magnetic activation of tissue along the long-axis of a ventricular septum;
a processing means for generating magnetic resonance (MR) images of a region of interest of the ventricular septum over the course of a plurality of heart beats such that a time-map of the MR images is obtained; and
a processing means for processing data from the time-map such that a beginning and an end of diastasis can be determined.

20. The system according to claim 19, including generating a position graph of a region of interest.

21. The system according to claim 20, including generating a velocity graph of the region of interest.

22. The system according to claim 21, wherein the velocity graph is generated by taking a first derivative with respect to time of the position graph of the region of interest.

23. The system according to claim 20, wherein the region of interest is near the base of the septum.

24. The system according to claim 20, wherein the region of interest spans a depth range of about 1 cm near the base of the septum.

25. The system according to claim 19, wherein the MR images are line images obtained by an MR pulse sequence.

26. The system according to claim 19, wherein the MR images are line images obtained by 2D excitations.

27. The system according to claim 19, wherein the MR images are generated during a breath hold.

28. The system according to claim 19, wherein the beginning and the end of diastasis are determined relative to an R-peak.

29. The system according to claim 28, wherein the R-peak is determined by using an ECG.

30. The system according to claim 19, where the start and end times of diastasis are determined by finding time points that mark the approach to and departure from, respectively, a low velocity time period in between the velocity peaks associated with early and late ventricular filling.

31. The system according to claim 19, where the start and end of diastasis is determined by finding a time period in between the velocity peaks associated with early and late ventricular filling where the absolute velocity is below a selected threshold.

32. The system according to claim 19, wherein the magnetic resonance image is generated from phase data obtained from the MRI.

33. The system according to claim 19, wherein the magnetic resonance image is generated from magnitude data obtained from the MRI.

34. The system according to claim 19, wherein diastasis is determined by intersecting the diastasis windows from multiple heartbeats.

35. The system according to claim 19, wherein diastasis is determined to be the period in between the average start and end times of the diastasis windows from multiple heartbeats.

36. The system according to claim 19, wherein heart rate is observed such that an observation of a predetermined threshold of heart rate change signals that a new timing of diastasis must be determined.
